(19) United States
(12) Patent Application Publication
(10) Pub. No.: US 2026/0325553 A1
(43) Pub. Date: Oct. 22, 2026

(54) Post COVID-19 Vaccine Remedies

(71) Applicant: Suzanne J. Paxton-Pierson, Ellensburg, WA (US)

(72) Inventor: Suzanne J. Paxton-Pierson, Ellensburg, WA (US)

(21) Appl. No.: 19/328,235

(22) Filed: Sep. 15, 2025

Related U.S. Application Data
(62) Division of application No. 17/482,364, filed on Sep. 22, 2021.
(60) Provisional application No. 63/179,170, filed on Apr. 23, 2021.

(51) Int. Cl. A61K 31/593; A61K 31/19; A61K 31/352; A61K 31/353; A61K 31/375; A61K 45/06

(52) U.S. Cl. CPC A61K 31/593; A61K 31/19; A61K 31/352; A61K 31/353; A61K 31/375; A61K 45/06

(57) ABSTRACT

Methods and compositions to lessen toxicities in COV19 vaccine aftermath. These involve administering synergistic or complementary formulations of early detoxification agents, antioxidants, anti-inflammatories, pro-immunity agents, gp41/gp120 inhibitors, nAChR/NAD⁺, boosters, ACE-2 modulators, anticoagulants and other micronutrients, vitamins, plant-derived compounds and selections from pertinent, major drug classes. tailored to address post-vaccination complications effectively.

There are urgent interventions needed to prevent untoward adverse genetic and toxic events and death in certain persons receiving the CV19 injections. Many aspects of the CV19 injection, its full complement of ingredients, binary interplay with EMF/RF, and toxicity etiology, at this time, remain unknown, tentatively known, or unclear.

On Oct. 22, 2020 CBER FDA published adverse event and toxicities from the CV19 vaccine injections including: Death; Guillain-Barre Syndrome; Acute Disseminated Encephalomyelitis; Transverse Myelitis (polio symptoms); Encephalitis; Myelitis: Encephalomyelitis; Meningoencephalitis; Meningitis; Encephalopathy; Convulsions; Seizures; Stroke; Narcolepsy; Cataplexy; Anaphylaxis, Acute Myocardial Infarction; Myocarditis; Pericarditis; Autoimmune Diseases; Miscarriages; Stillbirths; Demyelinating Diseases; Non-Anaphylactic Allergic Reactions; Thrombocytopenia; Disseminated Intravascular Coagulation; Venous Thromboembolism; Arthritis; Arthralgia; Joint Pain; Kawasaki Disease; Multisystem Inflammatory Syndrome in Children (MIS-C); Vaccine Enhanced Disease; Antibody Dependent Enhancement; Pathologic Priming] as well as Neurological Diseases including Spongiform Encephalopathy, Early Onset Dementia and Alzheimer's, Solid Tumors, Blood Cancers. Many others resultant diseases are as yet to be discovered and no long-term safety data exist. Post CV19 injection symptoms may be caused by singular ingredients or various ingredients or in conjunction with external radiation sources which may cause synergistic toxicities. Many ingredients may not be listed on a legal, official package insert as yet. These ingredients may at least include: mRNA or adenovector routes to production of the synthetic S1 antigen spike protein, heavy metals such as barium, chromium, tungsten, and excess iron, graphene oxide in synthetic PEG, and potentially infective agents such as Trypanosoma cruzi. All of the ingredients once introduced into the body may also self-assemble and interplay with EMF or ionizing radiation in the environment.

Prevention of mRNA translation via destruction or disabling of the mRNA or adenovector DNA; the amount of toxic spike protein production may be inhibited or stopped. S1 spike protein shedding also causes “occupational secondary adverse events” to persons in proximity or exposed to bodily fluids such as breast milk or blood or organs. Compassionate use ivermectin and hydroxychloroquine should be made available to the public for free or cost. mRNA and adenovector DNA are sensitive to heat, therefore immediate local heat treatments within about 8 inches of the injection, particularly above about 37 C when employed, would denature the genetic payload. Localized release-bleeding and suction can remove some degree of injection contents. Sclerants such as amphiprotic sodium tetradecyl sulfate or polidocanol and others can restrict movement of contents peripherally. Natural product detergents such as soyasaponin or sapinogen, licorice saponin or sapogenol or others violate the lipid micelle structures, exposing the genetic payload for reduction. Binding of cationic lipid nanoparticles to prevent DNA binding and damages with phospholipids (negatively charged) lecithin, which contains choline phosphate plus glycerol, reduces cell entry. Protease inhibitor—reverse transcriptase inhibitors may be employed. Halting DNA synthesis temporarily can prevent DNA alterations by intercalation of adenovector payloads. Protection of DNA from mRNA reverse transcription and vector chromosomal integration may be facilitated by halting DNA synthesis temporarily in order to prevent DNA alterations from adenovector payload or retroviral material which may be reverse-transcribed by intrinsic reverse transcritpases. Sulfhydryl proteolytic enzymes such as bromelain and papain may inhibit or stop, temporarily, DNA synthesis, which restarts in 48 hours at an accelerated rate. This method is exploitable for preemptive radioprotection as well. Pineapple's bromelain is a proteolytic enzyme extract from pineapple stems. Aminothiols alter chromatin structure and also inhibit DNA synthesis, causing observed post-treatment inhibition of DNA formation and subsequent blockage of cells in late S or G2 phases. Polyamine depletion post-gene therapy may improve DNA repair fidelity. Polyamine depletion stalls DNA synthesis (70-80%) as shown by DFMO-treated cells in which DFMO inhibits ODC, the first enzyme in polyamine biosynthesis. Cysteamine inhibits DNA synthesis in mouse bone marrow. WR-1065 can distort DNA supercoiling and this perturbation could stall DNA synthesis allowing for less intercalation. WR-2721 has close structural similarity to the polyamines spermine and spermidine, which implicates its effects on DNA synthesis, DNA repair and genomic stability. WR-2721 and its metabolites compete with putrescine for uptake into rat lung slices via the polyamine transport system. Both polyamines and aminothiols stabilize DNA and affect chromatin structure and condensation. (Aminothiols may also bind and stabilize DNA which is not covered by histones. WR-1065 can distort DNA supercoiling and this perturbation could stall DNA synthesis. Thymoquinone and other Nigella sativa seed constituents through inhibition of cell growth in G1 phase allow inhibition of DNA synthesis. Nigella sativa oil injected in mice protects normal tissues and prevent spike protein damages. Zinc is a reverse transcriptase inhibitor and quercetin and hydroxychloroquine or chloroquine serve as ionophores for zinc. Diallyl sulfide inhibits nuclear aberrations possibly from mRNA or pDNA; its protection effects are dose-dependent but must be dosed prior to CV19 shots. DAS dosed pre-injection will suppress DNA synthesis and ODI activity (which regulates DNA synthesis); both DNA synthesis and ODI activity are normally elevated after radiation or damages/alterations. DFMO abolished DAS activity to radio-protect against colonic nuclear damage; thus DAS protects via a polyamine-related pathway. Dry fasting and electrolyte fasting reduce DNA alterations and spike protein manufacture.

The S1 antigen spike protein ACE2 receptor binding domain causes destruction of ACE2 receptors. ACE-2 inhibition results in lower angiotensin II. ACE2 is inhibited by ACE2 residues; Q24, D30, H34, Y41, Q42, M82, K353 and R357; a high lysine diet with heightened arginine reduces ACE2 affinity and interferes with fusion domain for S1 spike protein by 10%; a more alkaline blood dimishes by 17% as achieved by sodium bicarbonate. L-lysine blocks L-arginine transport in vitro, via competitive blockade and down-regulation of cationic amino acid transporters. Dose of up to 3 g/day lysine very safe, up to 10-15 g/day (which may result in diarrhea). ACE2 destruction may lead to mitochondrial ablation, causing CHF, stroke, and pulmonary hypertension. Suramin, and pine needle extract may limit mitochondrial destruction. Increased G and C in the mRNA increases ACE2 protein receptor affinity over a thousand fold. Ivermectin blocks ACE2-receptors. Angiotensin receptor blockers block the receptor and protect it from spike protein destruction, which may result in greater survival. A reduction of mortality results from using ACE2-inhibitors/ARBs or means to upregulate ACE2 in CV. ACE-I and ARBS in post injection in a patient may upregulate ACE2 enzyme which is protective. Blocking of ACE2 receptors from spike protein damages may help preserve health. The S1 spike protein pharmacokinetic data show that it is produced ubiquitously, with uptake into spleen, bone marrow, ovaries, testes, lungs and brain. The S1 antigen alone was shown to cause all COVID19 symptoms. L-carnosine, vitamin D3, melatonin reduce spike protein ACE2 toxicity; dry fasting and electrolyte fasting reduce spike protein production.

mRNA when used for cancer treatments have shown activated oncogenes activated and tumor suppressor genes turned off, and the injection may cause lowered p53 apoptosis. Dry fasting or electrolyte fasting, sauna use, enhance heat shock proteins and p53 apoptosis and autophagy; HCQ and fenbendazole have anticancer properties. Heavy metal analyses have shown the presence of chromium, barium, tungsten, and iron. EDTA and sodium ferrocyanide can assist in clearing heavy metals which may cause oxidative and other damages. Sodium ferrocyanide has shown a similarly low oral toxicity with an LD50 in rat at 980 mg/kg (Chemical Abstract Service data for sodium ferrocyanide). Copper (II) ferrocyanide on mesoporous silica had greater affinities for Cs and Tl than Prussian Blue, and it was less affected by the solution pH, competing cations, and matrices. (Selective Capture of Cesium and Thallium from Natural Waters and Simulated Wastes with Copper Ferrocyanide Functionalized Mesoporous Silica Thanopon, S. J Hazard Mater, Oct. 15, 2010; 182(1-3): 225-231). Sodium ferrocyanide USP is a potentially more widely available, higher-affinity extreme-low toxicity orally-dosable alternative chelation agent compared to others.

Inflammatory cytokines, higher phospholipase A2 and ROS commensurate with the CV19 injection will be aided by curcumin/turmeric, C60, vitamin C, other antioxidants, and honey. Guillain Barre is linked to inflammatory cytokines. The tat protein calms GBS and turmeric duplicates the effect. Queensland University CV19 injection patients became HIV-positive: GBS can be the first sign a patient is HIV-positive. Guillain-Barré Syndrome (GBS) is an acute demyelinating polyneuropathy which can occur post-infection. Several viral infections have been associated with GBS including cytomegalovirus (CMV), Hepatitis E virus (HEV) and Zika virus. Triumeq, a once daily tablet combination of the antiretrovirals abacavir, lamivudine and dolutegravir has proven useful in HIV+GBS patients. CMV is a latent herpes DNA virus with retroviral components which causes retinal blindness. EBV is a latent herpes DNA cancer virus which awakens dormant HERV-W, a retrovirus. Many naturally derived anti-HIV compounds are flavonoids, coumarins, terpenoids, alkaloids, polyphenols, polysaccharides or proteins. Natural product neurologically-active gp41 fusion inhibitors include theaflavin, theaflavin-3-gallate, theaflavin digallate, epitheaflavin-3V-gallate, gallocatechin gallate, epigallocateching gallate EGCG, epigallocatechin 3,4 digallate and 2,2-bisepigallocatechin digallate. Cistus incanus extract and cinnamon-derived procyanidin compound type A polyphenol trimer, particularly A-type procyanidin oligomers, displays anti-HIV-1 as a gp120 inhibitor for co-receptor-binding sites on gp120. An extract from Spirulina, has documented antioxidant, immunomodulatory, anti-inflammatory and antiviral activities. Other RTase anti-HIV compounds are anti HIV alstonine-HCl, arctigenin, baicalein, baicalein, berberine, columbamine, digallic acid, ellagic acid, ellipticine, fagaronine-chloride, gossypol, jatrorrhizine, kaempferol, nitidine, o-methyl-ellipticine, punicacortein, punicalin, quertagetin, quercetin, and trachelogenin. Resultant immunodeficiencies post injection such as CD8 and comprehensive antibodies may be aided by vitamin D3, 25-OH vitamin D and calcifediol (soluble in DMSO and ethanol) which have been associated with lower mortality. Honey, ashwaghanda and mushrooms can improve immunity. Lentinan (Shiitake) compounds are antiviral, immunostimulant and antitumor. Whole Shiitake mushroom may be used as food or taken as tea. Lentinan (1,3 β-D-glucan) is a polysaccharide isolated from shiitake mushrooms which has anti-cancer effects in colon cancer and leukemia cells. Lentinan has anti-fungal and anti-HIV-1 reverse transcriptase activity. Lentinan has anti-fungal and anti-HIV-1 reverse transcriptase activity. Lentinan is antiproliferative, immunostimulatory, hepatoprotective, antimutagenic, and increases CD4 and neutrophil activity. Shiitake improves quality of life and survival in various cancer patients. Mushroom extracts are pro-immunity such as Ganoderma lucidum (Reishi), Lentinus edodes (Shiitake), Inonotus obluquus (Chaga), Maitake. Shiitake compounds are antiviral, immunostimulant and antitumor. Chaga increases tolerance to radiation. Chaga mushroom reduced toxicity associated with radiation in animal models. IVMN tells functional adapative T cells how to act. Antibody dependent enhancement (ADE) or pathogenic priming results from spike protein expression on the cell surface and autoimmune attack upon re-exposure to wild type virus, along with overall comprehensive antibody titer flattening with subsequent boosters. Improvement of natural immunity to restore reactivity will be preferential to boosters. Treatment for coronaviruses with hydroxychloroquine, chloroquine, ivermectin, with dexamethasone or budesonide and other anti-inflammatories such as turmeric/curcumin or NSAIDS such as naproxen, aspirin, ibuprofen, etc. are of some benefit for cytokine storm. The spike protein surface expression alters capillary endothelium interaction with platelets which interpret the roughness as damages and react in coagulation cascades particularly with damaged RBC. Red blood cell morphology changes with the injections, either destroying cells, causing stacking, or clumping. Hydration, exercise, anticoagulants are important to preventing clots. D-dimer and fibrinogen tests for microclots stear treatments to occult nascent tissue damages or tissue death; troponin tests can measure cardiac and skeletal muscle death. Treatment with resveratrol and vitamin B3 replaces some destroyed cells with adult stem cells. Destruction of mitochondria leads to lethargy, CHF, lack of vitality. Nicotinamide mononucleotide is a precursor to NAD+, useful to gain more cell energy; caffeoylquinic acid, vitamin D3 with C60 reduces ROS can help mitochondrial health to produce normal ATP. If the synthetic spike protein syncytin homology is expressed, this is pro-cancer. IVMN and licorice are antisyncytia compounds. 18β-GA inhibits viral attachment, internalization, and by stimulating IFN secretion. Ingestion of spirulina or of spirulina extracts enriched in phycocyanobilin may have potential for boosting type 1 interferon response in the context of RNA virus infection. Possible protozoal contamination with Trypanosoma cruzi is treatable with pentamidine (aerosolized or not) or zidovudine; to kill the parasite, Chagas disease is treated with benznidazole and also nifurtimox, especially effective (nearly 100%) in curing if given soon after infection by injection at the onset of the acute phase. Zinc is able to up-regulate host immune response against Trypanosoma cruzi replication. The noraporphine alkaloid (−) caaverine and piperine have shown antiprotozoal activity against T. cruzi parasites. Hydroxychloroquine, ivermectin, fenbendazole, oxytetracycline, doxycycline, amoxicillin, clindamycin, zinc 25 mg, sodium bicarbonate and borax. Both borax and boric acid are known as borates, which are compounds that come from the element boron, are used to detox at about 150 ppm. Spirochete bacterium Borrelia burgdorferi and rarely, Borrelia mayonii, due to symptoms of acute onset of fever, headache, myalgia, arthralgia, and bullseye rash, may also be treated with doxycycline, amoxicillin or cefuroxime. Prions are replicated, misfolded proteins in the spike protein which disrupt the FUS gene causing ALS or cancer. The Frontal Temperal Lobe FTD43 protein FTL degenerates and causes early Alzheimer's and dementia and 5 kinds of neurological diseases whereby the patient loses speech and facial recognition. EOD is seen in ages 45-50 called early onset dementia. Epigallocatechin gallate and heat shock protein (which spikes with dry fasting and saunas) may limit dementia and Alzheimer's. The synthetic PEG-graphene oxide technology of fatty lipoid liquid nano-crytals as carriers which encase the genetic payload forms a sturdy LDL-like pseudovirus lipid nanoparticle micelle. PEG poses an anaphylaxis risk and lung toxicity risks. It is prudent to have epinephrine, norepinephrine, noradrenaline, and beta agonists on hand. While the spike protein attaches to ACE2 receptors, based on the clinical observation of lower prevalence of smoking in hospitalized CV19 patients, the RBD of the S1 spike protein binds to nicotinic acetylcholine receptors. There is a toxin-like amino acid sequence in the receptor binding domain of the S1 spike protein (aa 375-390) homologous to neurotoxic homolog NL1 (which is a snake venom toxin known to interact with nicotinic acetylcholine receptors (nAChRs) whereby nicotine and other nicotinic cholinergic agonists such as NAChR neuronal agonists such as cytisine of Fabaceae family, anatoxin-a from cyanobacter anabseine, d-tubocurarine Chododendron tomentosum plant, coniine of Conium maculatum plant, candicine, may protect nAChRs and thus have therapeutic value in CV19 vaccine injection patients.

Graphene oxide nanoparticles are an undisclosed adjuvant and excipient (ALC0159) in at least some of the CV19 injection/vaccine formulations. Purging or disabling of SPION magnetofecting poisonous graphene oxide and associated assembly ferrous ferric oxide is possible via chelating agents including sodium ferrocyanide, EDTA, strong magnetic forces, surgical removal, nicotine, ethanol, vitamin C, glutathione or glutathione precursors N-acetyl cysteine, selenomethionine, sodium selenite, C60 antioxidant, borax, nanosilver or silver colloid, and turmeric. Graphene oxide acts as a superconductor and EMF repeater (neural lace or neuralink), it may interact with up through 3G, 4G, gigahertz and soft-x-ray or other ionizing radiation frequencies and interface neuronal and physiological output/input with external feeders as micro receiver antenna for low frequency radiation or pulsed high frequency, and as readers. The graphene oxide multiplies body EMF up to 400-fold, the resonant absorption being in the 5G range of about 41.6 gHz whereby graphene oxide heats and sparks. SPIONS (super paramegnetic iron oxide nanoparticles) cause magnetofection of DNA into the nucleus. The shot 5G optogenetic magnetoproteins in liquid crystal form can embed in the brain, which ostensibly could be used to control the brain. Magnetoproteins in the striatum may cause release of DA in conjunction with low frequency pulsed gigahertz. Magnetic liquid crystals in the bloodstream may behave as microreceivers of low frequency EMF with documented neurological consequences (such as zebrafish coiling behavior and dopaminergic reward bias behavior or as mentioned in Rwanda). Magnetoproteins have already been used to regulate blood glucose levels in mice. Nerve cells may be genetically engineered to become sensitive to radio waves and magnetic fields, by attaching them to an iron-storing protein called ferritin, or to inorganic paramagnetic particles. Injection positions anecdotally have been shown to possess both ferrous and magnetic properties.

Other useful compounds, ursolic acid, betulinic acid, betulin, chlorogenic acid, caffeic acid, neridienone, oleandrin, neriursate, kanerodione, kanerocin, uvaol, ursolic acid, alpha-amyrin, beta-sitosterol, campestrol, choline, diacetyl-nerigoside, quercetin, quercitrin, rosaginin, rutin, stearic acid, stigmasterol, oxyresveratrol, saikosaponin b2, tangeretin, nobiletin, glycyrrhizic acid, quercetin 3 rhamnoside, samarangenin, tetranortriterpenoid 1-cinnamoyl-2, 11-dihydroxymeliacarpin, pterocarnin A, chalepin, pseudane IX, manassantin B, dicaffeoylquinic acids, scopadulcic acid, trihydroxy-8-methoxyflavone, baicalin, glycyrrhiza chalcones, dammarenolic acid, ajoene, allicin, allyl methyl thiosulfinate, methyl allyl thiosulfinate, anthraquinone aloe emodin, triterpene saponin, diterpene aandrographolied, neoandrographoliede and 13-deoxy-11,12-didehydroandrographolide, sulfated polysaccharides, quercetin derivatives, lectins of Cananaviolia ensiformis, anthraquinones of Cassia angustifolia, acidic polysaccharides of Cedrela tubiflora, phenolic compounds of Cicer arietinum, catechin, epicatechin and B-type procyanidins of Cocoa nucifera, alkenes and amides of Echinacea purpurea, polysacchardic, choric acid and others of Echinacea purporea, sesquiterpene sclerocarpic acid of Glyptopetalum sclerocarpum, oligomeric to polymeric proanthocyanidins.

Other antihistamines: loratadine, hydroxyzine, carbinoxamine, cryproheptadine, desloratidine, livostin, levocetirizine, brompherinamine, ceterizine, chlorpheniramine, clemasatine, diphenhydramine, fexofenadine.

Other steroid type anti-anaphylaxis compounds: budesonide, dexamethasone, anti-rejection, immunosuppression medicaments including corticosteroids hydrocortisone, cortisone, ethamethasoneb, prednisone, prednisolone, triamcinolone, methylprednisolone, fludrocortisone, fluticasone.

Other beta receptor type anti-anaphylaxis compounds: albuterol, formoterol, etc. salmeterol, vilanterol, levalbuterol, olodaterol.

Other adrenergic-type anti-anaphylaxis compounds: epinephrine, dobutamine, norepinephrine, pseudephedrine, phenylephrine, oxymetazoline, methyldopa, clonidine, and dexmedetomidine, mirabegron, norepinephrine, dopamine, and isoprenaline.

Doses are in the therapeutic 0.0001% to 95% w/w or w/v range.

A method for mitigating negative effects of COVID19 “vaccines,” inoculations, immunizations, shots, jabs or injections, comprising administering to a human or mammal subject in need thereof, of a therapeutic core composition comprising a synergistic, therapeutically-effective amount of 18-beta Glycyrrhetinic Acid, Epigallocatechin Gallate, Spirulina extract, Cinnamon extract, Curcumin, Betulinic Acid, Vitamin D3, Zinc Ascorbate, Hesperitin, Luteolin, with optional Pentamidine and oral, nontoxic Sodium Ferrocyanide, and nicotinamide mononucleotide.

The method of claim 1 with the option of at least one or more of the following physical or drug treatments post-COVID19 “vaccine” exposure:
one or more in a composition of histamine type anti-anaphylaxis compounds including anti-IgE, antihistamine medicaments including loratadine, hydroxyzine, etc. and
one or more in a composition of steroid type anti-anaphylaxis compounds including budesonide, dexamethasone, etc. and
one or more in a composition of beta receptor type anti-anaphylaxis compounds including beta receptor medicaments albuterol, formoterol, etc., and
one or more in a composition of adrenergic-type anti-anaphylaxis compounds including, epinephrine, dobutamine, etc., and
application of stricture or tourniquet to isolate injection area from the rest of the body, optimally within the first minutes after injection, and
causing of localized release-bleeding near the injection site with vacuum local suction, and
one or more in a composition of decorporation agents including activated charcoal, ammonium perchlorate, Attapulgite, Bentonite clay, calcium alginate, chlorella, ethylenediaminetetraacetic acid, French Green clay, Fuller's Earth, green tea extract, Montmorillonite clay, Pascalite, psyllium husk powder, citric acid, calcium citrate, and Zeolite, sodium silicate, potassium silicate, silica, silicon dioxide, and
application of high thermal temperature optimally within the first minutes or hours after injection, of up to about 135° F. for up to about 8 seconds, up to 125° F. up to 1 minute and up to about 120° F. up to about 8 minutes, as well as lower temperatures above 105° F. for longer durations, local heat treatments directly upon and within about 8 inches of the injection, particularly above about 37° C., and
one or more in a composition local amphiprotic sclerants within optimally about up to 2 hours post injection at the same or nearby site, comprised of up to about 10 ml of 1% sodium tetradecyl sulfate or up to about 15 ml 0.5% polidocanol, and
demagnetization by subjecting the affected area or mammal to a strong field, pulsed or by alternating current demagnetization, or applying a reversed magnetic field to any magnetized tissues, and
one or more in a composition natural product detergents locally at the same or nearby site, comprised of injection with soyasaponin or sapinogen, licorice saponin or sapogenol, and
administration of metal chelators comprised of sodium ferrocyanide, or EDTA administered by slow IV infusion, and therapeutically-effective amounts of mint, polyphenols, flavonoids, rose hips, alpha-lipoic acid, N-acetyl cysteine, and
reduction in EMF effects by cloaking with EMF-blocker materials, proximate use of EMF absorbers, use of copper coils, ashwagandha, metformin for glucose control, and
one or more in a composition of anti-anaphylaxis, anti-inflammatory, anti-cytokine, antiprostaglandin medicants of benefit for cytokine storm including NSAIDS such as naproxen, acetyl salicylic acid, glabridin, ibuprofen, meclofenamate sodium, diflunisal, tolmetin, ketoprofen, flurbiprofen, ketorolac, tromethamine, indomethacin, fenoprofen, diclofenac, celecoxib, omega-three fatty acids, or hydroxychloroquine, chloroquine, ivermectin, and turmeric or curcumin, and
one or more in a composition of antiretroviral protease inhibitor medicaments abacavir, lamivudine and dolutegravir, atazanavir, darunavir, fosamprenavir, indinavir, lopinavir, ritonavir, nelfinavir, pentamidine (aerosolized or not), zidovudine; glycyrrhizin, glycyrrhetinic acid, betulinic acid, quercetin, salicin, camostat mesylate, and silybin and antisyncytium glycyrrhetinic acid, ivermectin, and berberine, and
one or more in a composition of a composition of anti-spike protein medicaments including hydroxy-chloroquine, chloroquine, quinine, artemisinin, Artemisia, fenbendazole, ivermectin, suramin, pine-needle extract, azithromycin, quercetin, vitamin D3, zinc, vitamin C, an alkaline high lysine diet with heightened arginine intake, sodium bicarbonate, melatonin, dry fasting, electrolyte fasting, and sauna use to reduce spike protein production, and
one or more in a composition of a composition of anti-CRISPR AcrIIA4 protein extracted from Listeria monocytogenes, and
administration of negatively-charged binder of cationic lipid nanoparticles lecithin, and
one or more in a composition of DNA synthesis halting agents sulfhydryl proteolytic enzymes bromelain and papain, DFMO, aminothiols amifostine, WR-2721, WR-1065, cysteamine, thymoquinone and other Nigella sativa extracts, and diallyl sulfide DAS (dosed pre-injection), and
one or more in a composition of one or more of a composition of antioxidants and omega three medicaments including C60, curcumin/turmeric, omega-three fatty acids, vitamin E, vitamin C, selenium, SOD, glutathione, flavonoids; flavonols; flavones; catechins; flavanones; polyphenols, anthocyanidins; isoflavonoids; and/or plant, vegetable, or fruit extracts including those obtained from green tree leaves, milk thistle, soybeans, wine grapes and their seeds, acai berry, coffee berry, feverfew, pomegranate, tropical ferns, turmeric, and witch hazel, EGCG, SOD, glutathione, vitamin D3, melatonin, GSH, ALA, beta carotene, catalase, GST, CoQ10, mint rose hips, vitamin A, famotidine, etc., N-acetyl cysteine, selenomethionine, sodium selenite, and
one or more in a composition of one or more of a composition of ACE-II receptor protection medicaments: kaemperfol, apigenin, quercetin, luteolin, emodin, chrysin, rhein, delphinidin, cyanidin, apigenin, rhoiflolin, rutin, quercetine, nicotianamine, catechin, epigallocatechin, epigallocatechin gallate, ferlic acid, chlorogenic acid, isoferulic acid, caffeic acid, delta-viniferin, myritilin, myricitrin, lactucopicrin 15-oxalate, nympholide afzelin, biorobin, phyllaemblicin B, baicalin, hesperitin, scutellarin, glycyrrhizin, curcumin, tangeretin, nobiletin, naringenin, brazilein, brazilin, galangin, and acetoxychavicol acetate, captopril, analapril, ramipril, trandolapril, perindopril, benazepril, fosinopril, temocapril, L-carnosine, silybin, and salicyl-carnisine, losartan, irbesartan, valsartan, azilsartan, candesartan, eprosartan, olmesartan, telmisartan, high lysine diet, high arginine diet, sodium bicarbonate, and
one or more in a composition of one or more of a composition anticoagulant compounds as needed including therapeutic amounts of water warfarin, coumarin, heparin, apixaban, dabigatran, edoxaban, enoxaparin, allicin, ajoene, and S-allyl cysteine, hydration, increased exercise, NSAIDS, and
one or more in a composition of one or more of a composition antiprotozoal compounds (and for associated infections) chloroquine, ivermectin, hydroxychloroquine and fenbendazole, nanosilver or silver colloid, pentamidine (aerosolized or not), zidovudine; Chagas disease treatments benznidazole and also nifurtimox, noraporphine alkaloid (−) caaverine and piperine, ivermectin, oxytetracycline, doxycycline, amoxicillin, clindamycin, doxycycline, amoxicillin or cefuroxime, zinc, sodium bicarbonate, borax at about 150 ppm, and
one or more in a composition of one or more of a composition immune enhancement compounds vitamin D3, and honey, mushroom extracts Ganoderma lucidum (Reishi), Lentinus edodes (Shiitake), Inonotus obluquus (Chaga), Maitake, Glycyrrhizae pro-interferon 18β-Glycyrrhetuinic Acid, interferon, and
one or more in a composition of one or more of a composition anti-HIV compound: gp41 fusion inhibitors theaflavin, theaflavin-3-gallate, theaflavin digallate, epitheaflavin-3V-gallate, gallocatechin gallate, epigallocateching gallate EGCG, epigallocatechin 3,4 digallate, 2,2-bisepigallocatechin digallate and gp120 inhibitor Cistus incanus extract, cinnamon-derived procyanidin compound type A polyphenol trimer, A-type procyanidin oligomers, and
antiviral extract from Spirulina, an antioxidant, immunomodulatory, anti-inflammatory and antiviral substance, and
one or more in a composition of antineurotoxicity and anti-GBS medicaments NAChR cholinergic neuronal agonists nicotine, cytisine, anatoxin-a, anabseine, d-tubocurarine, coniine, C60, curcumin, turmeric, and
one or more in a composition of a composition pro-mitochondrial medicaments nicotinamide mononucleotide is a precursor to NAD+, NMN, vitamin D3, caffeoylquinic acid or cinnamic acid, C60 to reduce ROS, and resveratrol.

1. A method for treating post-COV19 vaccine patient, comprising administering zinc at a therapeutically effective amount of 25 mg/day.

2. A method for treating post-COV19 vaccine patient, comprising administering lysine at a therapeutically effective amount of 3 g/day.

3. A method for treating post-COV19 vaccine patient, comprising administering zinc at a therapeutically effective amount of 25 mg/day and lysine at a therapeutically effective amount of 3 g/day.
